(19) United States
(12) Patent Application Publication
(10) Pub. No.: US 2020/0171085 A1
(43) Pub. Date: Jun. 4, 2020

(54) Method of Treating Respiratory Tract Infection

(71) Applicant: The Hospital for Sick Children, Toronto (CA)

(72) Inventors: Theo Moraes, Toronto (CA); Martin Post, Toronto (CA); Yuchen Cen, Toronto (CA); Michael Litvack, Toronto (CA)

(21) Appl. No.: 16/614,994

(22) PCT Filed: May 18, 2018
(86) PCT No.: PCT/CA2018/050590

§ 371 Date: Nov. 19, 2019

Related U.S. Application Data
(60) Provisional application No. 62/508,642, filed on May 19, 2017.

(51) Int. Cl. A61K 35/15; C12N 5/0786; A61K 9/00; A61P 31/14

(52) U.S. Cl. CPC A61K 35/15; A61P 31/14; A61K 9/007; C12N 5/0645

(57) ABSTRACT

A method of treating a respiratory viral infection in a mammal is provided. The method comprises administering to the mammal a therapeutically effective amount of alveolar-like macrophages, or an anti-viral factor produced by alveolar-like macrophages.

### FIELD OF THE INVENTION

The present invention generally relates to respiratory viral infections, and in particular, to methods of prophylaxis and treatment of such infections.

### BACKGROUND OF THE INVENTION

Respiratory syncytial virus (RSV) is the leading cause of lower respiratory tract infection in young children and infants worldwide and is an important cause of morbidity and mortality in elderly and immunocompromised populations. The World Health Organization (WHO) reports 64 million cases and 160,000 deaths each year due to RSV. Nearly all children will be infected by the age of 2, of which one third will develop lower respiratory tract disease and 2.5% will be hospitalized. Those infected by RSV remain susceptible to reinfection throughout life as sterilizing immunity does not occur. It is unclear why children, elderly and immunocompromised populations are at a higher risk for severe disease; however, an immune pathological mechanism has been suspected. RSV pathology is primarily mediated by virus induced airway epithelial cell dysfunction and induction of an immune inflammatory response associated with development of bronchiolitis and pneumonia. Inflammation from infection must be carefully balanced to eliminate the infection and prevent excessive tissue damage. Currently there is no vaccine or specific anti-viral treatments against RSV.

RSV primarily infects airway epithelial cells stimulating the Toll-like receptors (TLR) 4, TLR2, TLR3 and the retinoic acid-inducing gene I (RIG-I) leading to the activation of pro-inflammatory genes. Stimulation induces chemokine recruitment of neutrophils, monocytes, dendritic cells, macrophages and T cells. Alveolar macrophages (AM) comprise 95% of the leukocytes in the airway and act as the initial responders to airway pathogens, initiating and maintaining the immunological response and clearing cellular debris. Several studies have shown immunocompromised mice with defective AMs, but competent natural killer (NK) and T cell function show enhanced RSV infection, increased inflammatory markers and worsening airway occlusion, similar to patients hospitalized with severe RSV infection. The studies indicate a protective role of AMs in the context of RSV. It has been theorized that AMs initiate an early inflammatory response against infection, limiting viral replication. As the infection progresses AMs are important for resolving the inflammatory response through clearance of inflammatory cellular debris, protecting against immunopathology.

It would be desirable to develop an anti-viral treatment useful against respiratory viral infection such as RSV.

### SUMMARY OF THE INVENTION

It has now surprisingly been determined that alveolar-like macrophages developed from stem cells, termed pluripotent stem cell derived alveolar-like macrophages (PSC-AMs), are useful to treat respiratory viral infections.

Thus, in one aspect of the present invention, a method of treating a respiratory viral infection in mammal is provided. The method comprises administering to the mammal alveolar-like macrophages, or one or more anti-viral factors secreted by alveolar-like macrophages.

In another aspect of the invention, the use of alveolar-like macrophages, or one or more anti-viral factors secreted by alveolar-like macrophages, to treat a respiratory viral infection.

In another aspect, an anti-viral factor is provided, which is produced by alveolar-like macrophages.

These and other aspects of the invention are described in the detailed description by reference to the following figures.

### BRIEF DESCRIPTION OF THE FIGURES

FIG. 1. PSC-AMs are not productively infected by RSV. PSC-AMs, HEp-2, and PBMC cells were exposed to RSV-GFP at an MOI of 1. Following exposure, GFP florescence was measured at 24 hour intervals for 72 hours. (a) HEp-2 cells were efficiently infected by RSV-GFP, PBMC cells are partially infected and PSC-AMs are not infected by RSV. (b) Nucleolin gene expression in PSC-AMs was shown to be decreased by 2 log-fold relative to primary murine AM. SD are plotted as error bars. Gene expression analysis was carried out at n=1, while (a) HEp-2 n=1 technical triplicate, PBMC n=1 technical triplicate, PSC-AM n=3 technical triplicate.

FIG. 2. PSC-AMs reduce RSV infectivity. S19 cells or PSC-AMs were co-incubated with RSV-GFP in suspension for 1 hour, and the viral titer in the supernatant was measured following incubation. (a) S19 cells conferred no significant change in viral titer compared to virus alone. (b) PSC-AMs confer a concentration dependent reduction in RSV infectivity. Cold conditions may inhibit macrophage function. (c) PSC-AMs kept at 4° C. did not inhibit viral infectivity. SD are plotted as error bars. Statistics were carried out using a 2way analysis of variance with Dunnet's correction for multiple comparisons against control RSV *p<0.05 **p<0.005 ***p<0.0005. Replicates were carried out as follows (a) n=3 technical duplicate (b) n=l technical duplicate (c) n=l technical duplicate.

FIG. 3. PSC-AMs increasingly reduce RSV infectivity over time. RSV was kept in EMEM at 37° C. with plaque assays performed at specific time intervals. (a) RSV infectivity was stable in media kept at 37 degrees for up to 4 hours with complete loss of infectivity by 24 hours. PSC-AMs (b) 2×10⁵ and (c) 1×10⁶ co-incubated with RSV between 1-4 hours showed increased reduction of viral infectivity over time. SD are plotted as error bars. Statistics were carried out using a 2way analysis of variance with Dunnet's correction for multiple comparisons against 1 hour time points *p<0.05 **p<0.005. ***p<0.0005. Replicates were as follows (a) n=2 technical triplicate (b,c) n=2 technical triplicate

FIG. 4. Viral-like particles associated with phagosome like structures within PSC-AMs. PSC-AMs (a) or PSC-AMs exposed to RSV for one hour (b,c) were fixed and viewed with transmission electron microscopy (TEM). (a) PSC-AMs contain many phagosome-like structures. (b) PSC-AMs contain viral like particles (arrows) associated with phagosome like structures. (c) 80000× magnification of a phagocytic-like event occurring.

FIG. 5. PSC-AMs confer anti-viral effects through a secreted factor. RSV exposed to UV radiation inhibits viral infectivity (a). PSC-AMs were cultured with RSV or UV-RSV for one hour. (b) The supernatant was extracted and UV-irradiated and used as conditioned media. Conditioned media was then added to HEp-2 cells with competent RSV-GFP and infection was measured by florescence. The x-axis represents the contents of the media. Control indicating HEp-2 cells with EMEM, media control indicates UV irradiated EMEM, UV-RSV (+) indicates media containing UV inactivated RSV, PSC-AM (+) UV-RSV (+) indicates conditioned media from PSC-AMs exposed to UV inactivated RSV, PSC-AM (+) RSV (+) indicated conditioned media from PSC-AMs exposed to RSV. Conditioned media from PSC-AM (+) RSV (+) confer a reduction in RSV infection, indicating the presence of an anti-viral secreted factor. SD are plotted as error bars. Statistics were carried out (a) unpaired parametric two tailed t-test with guassian distribution *p<0.05 (b) using a 2way analysis of variance with Turkey correction for multiple comparisons *p<0.05 ***p<0.0005. Replicates were as follows (a) n=l technical duplicate (b) n=3 with at least a technical duplicate per sample.

FIG. 6. Prophylaxis ALM therapy protects against RSV pathogenesis. (a) Pictoral diagram representing the experimental set-up. (b) Body weights measured between intra-tracheal administration of ALMs or fibroblasts and RSV inoculation showed no significant differences. (c) Body weights measured following RSV inoculation showed a trend of weight loss peaking at day 3 in untreated groups. ALM instilled mice have reduced body weight loss. (d) Lungs were homogenized and assayed for RSV viral tires by plaque forming assay over HEp-2 cells. NIH3T3 instillation did not affect viral titres. ALM instillation significantly reduced viral titres. (e) Representative sections from lungs of RSV infected mice taken day 4 post infection (20× magnification). Left image from a mouse that received RSV while right is from a mouse that received ALMs 2 days prior to RSV.

### DETAILED DESCRIPTION OF THE INVENTION

In one aspect of the invention, a method of treating in a mammal a respiratory viral infection is provided. The method comprises administering to the mammal alveolar-like macrophages, or an anti-viral factor secreted by the macrophages.

Respiratory viral infection is used herein to refer to a viral infection of the respiratory system, including the upper respiratory system, e.g. from the sinuses to the vocal chords, and the lower respiratory system, from the vocal chords to the lungs. Viruses from various families may infect the respiratory system. Examples include, but are not limited to, viruses of the Adenoviridae family, e.g. Adenovirus sp.; viruses of the Parvoviridae family, e.g. Bocavirus sp.; viruses of the Coronaviridae family, e.g. Coronavirus sp. (the SARS virus); viruses of the Picornaviridae family, e.g. Enterovirus sp. and/or Rhinovirus sp.; viruses of the Pneumoviridae family, e.g. Respiratory syncytial virus and/or human metapneumovirus; viruses of the Orthomyxoviridae family, e.g. Influenza virus; and viruses of the Paramyxoviridae family, e.g. Paramyxovirus sp., which includes the parainfluenza viruses, e.g. human parainfluenza virus types 1 to 4 (HPIV-1, HPIV-2, HPIV-3 and HPIV-4) and the mumps virus; Pneumovirus, which includes respiratory syncytial virus; and Morbillivirus sp., which includes the measles virus.

The term “alveolar-like macrophage” refers to non-naturally occurring macrophages, generated in vitro from hemangioblasts prepared from pluripotent stem cells (PSCs), and which express markers expressed by naturally occurring alveolar macrophages, including one or more of F4/80 (mouse)/EMR1 (human), CD11c, SiglecF (mouse), CD80, CD86, CD206, CD11b, CD68, CD45 and SIRPα and have a capacity for uptake of acetylated low density lipoproteins (AcLDL). Alveolar-like macrophages are described, and may be prepared, according to the disclosure of WO 2016/127259 (PCT CA2016/050128), the contents of which are incorporated herein by reference. For example, a PSC-derived macrophage may be cultured in an alveolar macrophage-inducing medium, e.g. a serum-free differentiation medium suitable for use with macrophages, under suitable conditions, and for a sufficient period of time, e.g. 5-8 days. The alveolar macrophage-inducing medium comprises Granulocyte-Macrophage Colony-Stimulating Factor (GM-C SF), also known as Colony Stimulating Factor 2 (CSF2), and optionally comprises Macrophage Colony-Stimulating Factor (M-CSF), also known as Colony Stimulating Factor 1 (CSF1). The alveolar macrophage-inducing medium may additionally comprise one or more of IL-3, IL-6 and SCF. The amounts of the growth factors in the alveolar macrophage-inducing medium will generally be amounts which stimulate generation of alveolar-like macrophages from myeloid macrophages, for example, an amount of GM-CSF of about 10-100 ng/ml, and optionally, an amount of M-CSF of about 10-100 ng/ml, or an amount of GM-CSF and M-CSF in a ratio ranging from about 1:10 to 10:1 GM-CSF to M-CSF. In one embodiment, GM-CSF and M-CSF are used in about a 1:1 ratio, such as about 10-50 ng/ml of GM-CSF to about 10-50 ng/ml of M-CSF, e.g. 20 ng/ml of GM-CSF to 20 ng/ml of M-CSF; IL-3 is in an amount in the range of about 10-100 ng/ml, IL-6 is in an amount in the range of about 1-50 ng/ml and SCF is in an amount in the range of about 10-100 ng/ml.

The formation of alveolar-like macrophages may be confirmed minimally by expression of markers commonly expressed by alveolar macrophages such as one or more of F4/80 (mice)/EMR1 (human), SiglecF (mouse), CD11c (human/mouse), CD68 (human), CD169 (human), CD163 (human), and uptake of AcLDL (mouse/human). Other identifying markers that may be used to confirm formation of alveolar-like macrophages include CD45, CD11b (a unique marker of alveolar-like macrophages not highly expressed by primary AMs), SIRPα, CD80, CD86 and CD206. Alveolar-like macrophages also exhibit significantly increased expression of genes such as the LSR and RUNx2 (e.g. at least a 2-fold increase in expression or more, e.g. a 3-5 fold increase in expression as compared to primary alveolar macrophages). The formation of alveolar-like macrophages may also be confirmed based on functional characteristics, such as phagocytic activity, e.g. take up of apoptotic material and bacteria, and binding of the lung innate immune collectin, SP-D. Alveolar-like macrophages are Myb-independent, and are able to attain airway residence. They can also be expanded in vitro for periods of days, months and years, a unique property that naturally-occurring alveolar macrophages do not possess.

Alveolar-like macrophages as described herein are useful to treat respiratory viral infection. The terms “treat”, “treating” or “treatment” are used herein to refer to methods that favorably alter the infection including those that moderate, reverse, reduce the severity of, or protect against (i.e. are prophylactic), the progression of an infection of the respiratory system. For use in such a treatment, a therapeutically effective amount of in vitro-derived alveolar-like macrophages is administered to a mammal in need of treatment. The term “therapeutically effective amount” is an amount of alveolar-like macrophages required to treat the disease that does not exceed an amount that may cause significant adverse effects to the mammal in need of treatment. Alveolar-like macrophage dosages that are therapeutically effective will vary on many factors including the nature of the condition and site/organ to be treated, the mammal being treated and the dosage form utilized for administration. Appropriate dosages for use in such a treatment include dosages sufficient to result in airway residence of administered in vitro-derived alveolar-like macrophages of at least about 10%, and preferably, an airway residence of greater than 10%, for example, at least 20%, 30%, 40%, 50% or greater. In one embodiment, the dosage of in vitro-derived alveolar-like macrophages useful to treat a lung disease or disorder may be a dosage in the range of about 10⁵ to 10¹⁰ cells, for a sufficient period of time to achieve treatment. The treatment regimen may include daily administration of alveolar-like macrophages, or dosages administered more or less frequently, e.g. multiple dosages per day, or on alternate days, weekly, etc. The term “about” is used herein to mean an amount that may differ somewhat from the given value, by an amount that would not be expected to significantly affect activity or outcome as appreciated by one of skill in the art, for example, a variance of from 1-10% from the given value.

Alveolar-like macrophages for use in the treatment of a respiratory tract infection in a mammal may be formulated for therapeutic use by combination with a pharmaceutically acceptable carrier. The expression “pharmaceutically acceptable” means acceptable for use in the pharmaceutical and veterinary arts, i.e. not being unacceptably toxic or otherwise unsuitable. As one of skill in the art will appreciate, the selected carrier may vary with the intended mode of administration. In one embodiment, alveolar-like macrophages may be formulated for administration to the respiratory tract, e.g. by infusion or injection into a mammalian airway, e.g. intra-tracheally or intranasally, and thus, are formulated as a suspension in a medical-grade, physiologically acceptable carrier, such as an aqueous solution in sterile and pyrogen-free form, optionally buffered or made isotonic. The carrier may be a carbohydrate-containing solution (e.g. dextrose) or a saline solution comprising sodium chloride and optionally buffered. Suitable saline solutions may include varying concentrations of sodium chloride, for example, normal saline (0.9%), half-normal saline (0.45%), quarter-normal saline (0.22%), and solutions comprising greater amounts of sodium chloride (e.g. 3%-7%, or greater). Saline solutions may optionally include additional components, e.g. carbohydrates such as dextrose and the like. Examples of saline solutions including additional components, include Ringer's solution, e.g. lactated or acetated Ringer's solution, phosphate buffered saline (PBS), TRIS (hydroxymethyl) aminomethane hydroxymethyl) aminomethane)-buffered saline (TBS), Hank's balanced salt solution (HBSS), Earle's balanced solution (EBSS), standard saline citrate (SSC), HEPES-buffered saline (HBS) and Gey's balanced salt solution (GBSS).

In another embodiment, alveolar-like macrophages may be formulated for administration by routes including, but not limited to, inhalation. In this regard, aerosol formulations may be prepared in which suitable propellant adjuvants are used.

Other adjuvants may also be added to the composition regardless of how it is to be administered, for example, stabilizers, anti-oxidants, preservatives and anti-microbial agents may be added to the composition to prevent degradation of the composition over prolonged storage periods.

Alveolar-like macrophages may be used in combination with therapies such as medication to treat fever, e.g. acetaminophen or ibuprofen; inflammation, e.g. ibuprofen; and may also be used in combination with other anti-viral therapies.

In another aspect of the invention, an anti-viral factor isolated from alveolar-like macrophages derived from pluripotent stem cells is provided. The anti-viral factor is produced on exposure of the alveolar-like macrophages to competent RSV, and confers a reduction in RSV infectivity against human cells, e.g. human epithelial cells such as, but not limited to, HEp-2 cells. This factor is not produced from alveolar-like macrophages exposed to UV inactivated RSV and is resistant to UV radiation.

Embodiments of the invention are described in the following specific example which is not to be construed as limiting.

### Example 1—ALMs Resistant to RSV

Methods and Materials: HEp-2 cells were obtained from ATCC and maintained in EMEM supplemented with 10% fetal bovine serum (FBS), 2 mM L-glutamine and penicillin-streptomycin solution. Sf9 cells (Invitrogen) were maintained at 27° C. in Grace's supplemented media (Invitrogen) containing 10% heat-inactivated FBS. PSC-AM cells were kindly provided by Michael Litvack and the Post Lab and were prepared and maintained as described by Litvack et al. (American Journal of Respiratory and Critical Care Medicine 193, 1219-1229 (2016)), the contents of which are incorporated herein by reference. The cells were grown in a humidified environment at 37° C. and 5% CO₂. Recombinant RSV strains expressing GFP (RSV-GFP) were kindly provided by Dr. M. Peeples. RSV was produced in HEp-2 cells as previously described (Tayyari F, Marchant D, Moraes T J, Duan W, Mastrangelo P, Hegele R G. Identification of a nucleolin as a cellular receptor for human respiratory syncytial virus. Nature Medicine. 2011 Sep.; 17(9):1132-35). Briefly, HEp-2 cells (70% confluent) in T175 flasks were infected with RSV in 10 mL serum free EMEM for 2 h at 37° C., media was then topped up to a total volume of 30 mL EMEM with 4% serum. After incubation for 4 days, the cell supernatant and cells were collected, snap frozen and thawed twice. Cell debris was pelleted by centrifugation and the supernatant was aliquoted, snap frozen and stored at −80° C. for experiments.

All viral titers were carried out by infecting HEp-2 cells with a serial dilution of the virus in 96-well plates. Infection was allowed to proceed for 24 h at 37° C. Cells were assessed by florescence microscopy for GFP expression to detect infected cells. RSV supernatant was UV irradiated at 1200 mj/cm² for 30 mins using a UV Stratalinker 2400 (Stratagene). To confirm inactivation, a fluorescence plaque assay was performed on UV-RSV and RSV supernatants. Cold inactivated macrophage experiments were conducted with macrophages first cooled to 4° C., and subsequently exposed to RSV while maintaining the temperature in a thermomixer with gentle agitation. PSC-AM exposure to RSV for time and concentration experiments were carried out with macrophages incubated at 37° C. with gentle agitation with experiment specific concentrations and duration. Centrifugation was carried out to separate macrophages at 1250 g for 3 minutes, and followed by removal of supernatant and immediate use in a plaque assay to determine viral titers as described above. During the conditioning assay, methods were the same as the PSC-AM exposure assay with the addition of UV irradiation of media which was then added in a 1:1 ratio with EMEM supplemented with serum to HEp-2 cells which were subsequently challenged with RSV-GFP. Florescence measurements were measured by a florescence microscope at 48 hours post infection.

Immunofluorescence—PSC-AM or HEp-2 cells were placed on a glass slide by Cytospin at 1000 g for 1 minute. Cells were fixed with 4% PFA in sterile PBS for 20 minutes followed by 2 washes of PBS. Cells were subsequently permeabilized with 0.3% Triton X diluted in sterile PBS for 10 minutes following 2×PBS wash. Cells were subsequently stained with antibody. RSV F protein staining was carried out with a 1:250 dilution of an IgG mouse anti-RSV clone 133-1H antibody conjugated with Alexa 488 (Endmillipore) for 1 hour. F4/80 staining was carried out with a 1:100 dilution of a IgG rat F4/80 BM8 monoclonal antibody conjugated to eFluor 660 (eBioscience) for 12 hours. Nucleolin staining was carried out with a 1:100 dilution polyclonal rabbit nucleolin-specific IgG antibody H-250 for 1 h followed with a goat rabbit-specific Alexa 594 (Invitrogen) secondary antibody (1:400 dilution). Images were taken with a Leica DMi8 confocal microscope and analyzed using Improvision Volocity software with minor brightness and contrast adjustment not exceeding 20% of original values.

Electron Microscopy—Cells examined by electron microscopy were fixed in 2.5% (wt/vol) glutaraldehyde in 0.1M phosphate buffer at pH 7.4 followed by 1% osmium tetroxide then dehydrated and embedded in Epon Araldite resin. Ultrathin sections were prepared and stained in uranyl acetate and lead citrate before viewing. All samples were examined on a JEOL JEM 1011 transmission electron microscope (JEOL USA, Peabody, Mass.).

### Results/Discussion

### PSC-AMs are not Productively Infected by RSV

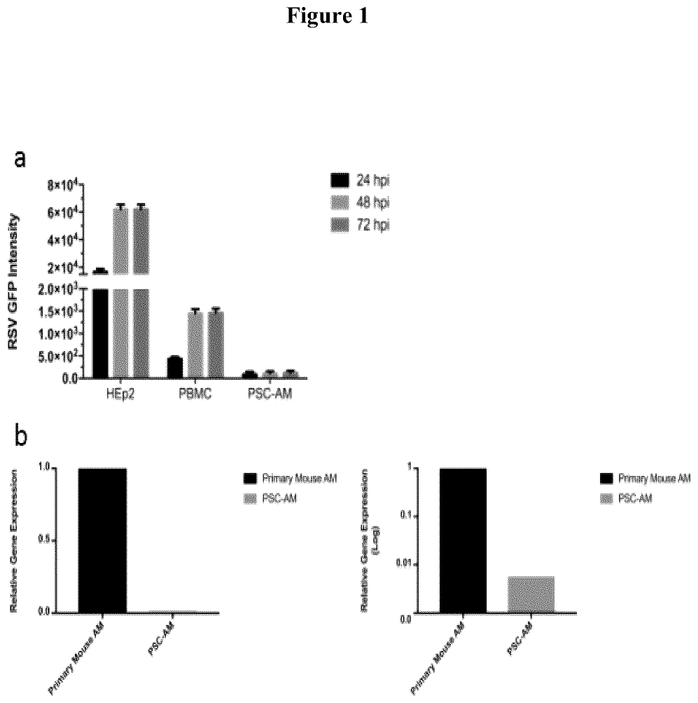

RSV primarily infects airway epithelial cells, and has also been shown to infect other cell types. To establish whether PSC-AMs play a role in RSV clearance, it was determined whether or not PSC-AMs can be productively infected by RSV. An RSV A2 strain expressing GFP (gift from Dr. M. Peeples) was used and GFP florescence was measured for viral infection. PSC-AMs, HEp-2, a human epithelial cell line and human peripheral blood monocytes (PBMC) were collected and exposed to RSV-GFP for 72 hours. As expected, HEp-2 cells were robustly infected by 24 hours and reach a peak infection by 48 hours post infection (hpi). PBMCs were similarly infected, however, viral infection was less efficient than in HEp-2 cells. PSC-AMs were not productively infected by RSV and after 72 hours no measurable GFP florescence was detected (FIG. 1A).

RSV utilizes the cellular receptor, nucleolin, to establish an infection. The expression of nucleolin in PSC-AMs was then characterized with respect to its potential mechanism for resistance. Gene expression of nucleolin was determined by PCR analysis comparing primary AMs derived from mice and PSC-AMs. PSC-AMs show a 100-fold decrease in the level of nucleolin gene expression when compared to mouse AM (FIG. 1B).

### PSC-AM Exposure to RSV Confers a Time and Concentration Dependent Reduction in Viral Infectivity

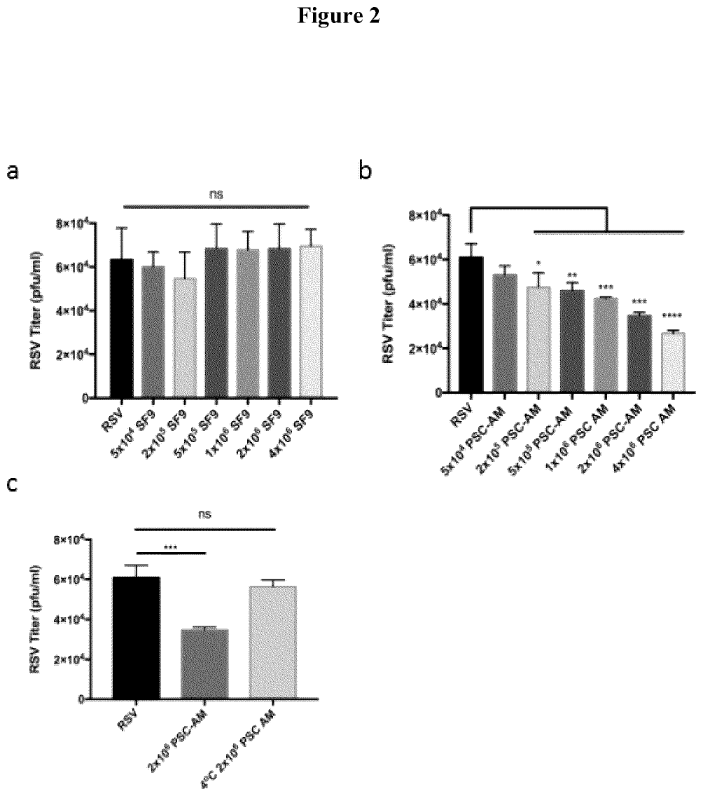

To study the direct interaction between PSC-AMs and RSV, PSC-AMs, S19 cells or media were co-incubated with RSV-GFP in a suspended culture. 519 cells are an insect cell line lacking nucleolin expression and are not infected by RSV. They were used as a cellular control in this assay. Following one hour of co-incubation, cells were pelleted by centrifugation and the supernatant was collected. A fluorescence plaque assay was carried out on the supernatant to determine RSV viral titres. A cell concentration range was established to determine concentration dependent effects. S19 cells did not affect viral titres across all concentrations tested (FIG. 2a). Co-incubation of RSV and PSC-AMs led to a dose-dependent reduction in viral titre (FIG. 2b). To investigate whether the loss of infectivity was due to a macrophage dependent effect, macrophages were co-incubated with RSV at 4° C. Cold incubation inhibits specific macrophage functions; phagocytosis is known to be inhibited while secretion of certain cytokines and exosomes may also be affected. PSC-AMs co-incubated with RSV at 4 degrees did not confer a significant reduction in viral infectivity (FIG. 2c). This indicates that at least part of the reduction in viral titre is dependent on a temperature sensitive macrophage function.

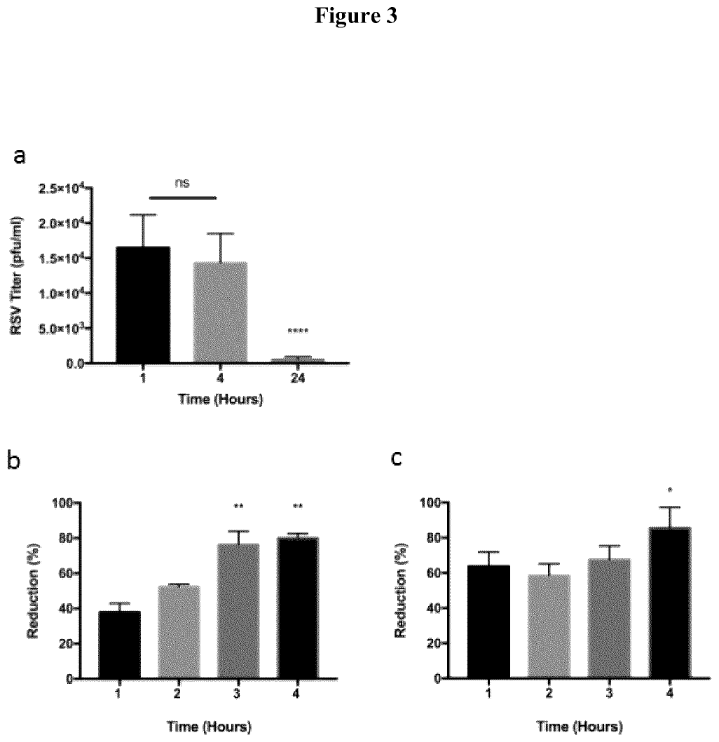

To further characterize the anti-viral properties of PSC-AMs, prolonged exposure to RSV was carried out in a time course to determine temporal effects. RSV is unstable in the environment and is known to lose infectivity over time when no host cell is present. To establish upper limits of the time course, RSV infectivity was determined over 24 hours. RSV infectivity was stable up to 4 hours, with infectivity lost by 24 hours (FIG. 3a). PSC-AMs were subsequently incubated with RSV for up to 4 hours. PSC-AMs co-incubated with RSV for 4 hours showed an increased reduction of RSV infectivity (FIG. 3b). Altogether it was found that PSC-AMs inhibit RSV infectivity in a time and dose dependent fashion due to a macrophage specific mechanism.

### PSC-AMs Exposed to RSV Internalize Viral Particles

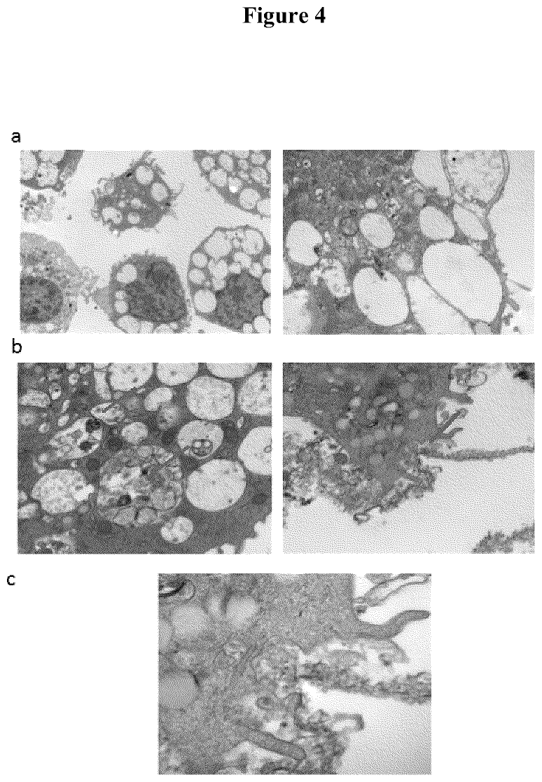

Following the observed reduction in viral infectivity when RSV was co-incubated with PSC-AMs, potential mechanisms were investigated. First, the possibility that the reduction in RSV infectivity was due to the phagocytosis or internalization of viral particles was considered. Electron microscopy (EM) and immunofluorescence (IF) staining was carried out to determine RSV localization within the macrophage. EM reveals viral-like particles found to be associated with phagosome-like structures within PSC-AMs (FIG. 4). To support these findings, immunofluorescence staining with antibodies against macrophage membrane protein F4/80, and a stain against the RSV fusion (F) protein identified viral proteins located within the macrophage confirming the observation with EM. Confocal microscopy Z-stacks indicated viral proteins located both at the surface and in the cytoplasm of PSC-AMs. This finding that PSC-AMs internalize RSV in the absence of RSV replication occurring is suggestive of an anti-viral mechanism.

### PSC-AMs Produce an Anti-Viral Secreted Factor

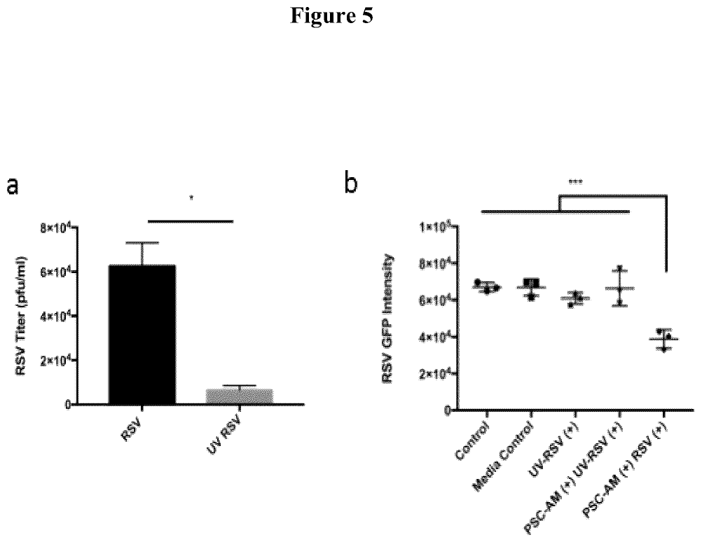

Next, the possibility that the PSC-AMs secrete an anti-viral factor was considered. To investigate the potential of an anti-viral secreted factor, a conditioned media assay was carried out. PSC-AMs were co-incubated with RSV, or UV-RSV. Previous studies have shown certain cytokines such as TNF are secreted by AMs in response to RSV challenge but not UV-irradiated RSV. UV irradiation was used to neutralize infectious viral particles (FIG. 5a). Following incubation, cells were separated by centrifugation and the conditioned media was collected. Conditioned media was subsequently UV irradiated to neutralize remaining infectious RSV virions. The conditioned media was added to HEp-2 cells simultaneous with competent RSV-GFP. RSV infection was measured by florescence intensity 48 hpi. HEp-2 cells were robustly infected by 48 hours in media. To control for potential changes in media proteins following UV irradiation, HEp-2 cells were incubated with UV irradiated media and no change in RSV infectivity was observed.

Subsequently, since UV irradiated RSV may confer protective responses from HEp-2 cells, media containing RSV alone that was UV irradiated was added as a control. UV-RSV did not confer a change in RSV infectivity. Conditioned media from PSC-AMs challenged with UV-RSV does not confer protection against viral infectivity with HEp-2 cells. Strikingly, conditioned media from PSC-AMs incubated with RSV confers a modest reduction in RSV infection (FIG. 5b). This indicates PSC-AMs in part control RSV infections through secretion of an anti-viral factor(s).

### Example 2—ALMs Provide Prophylaxis Protection Against RSV In Vivo

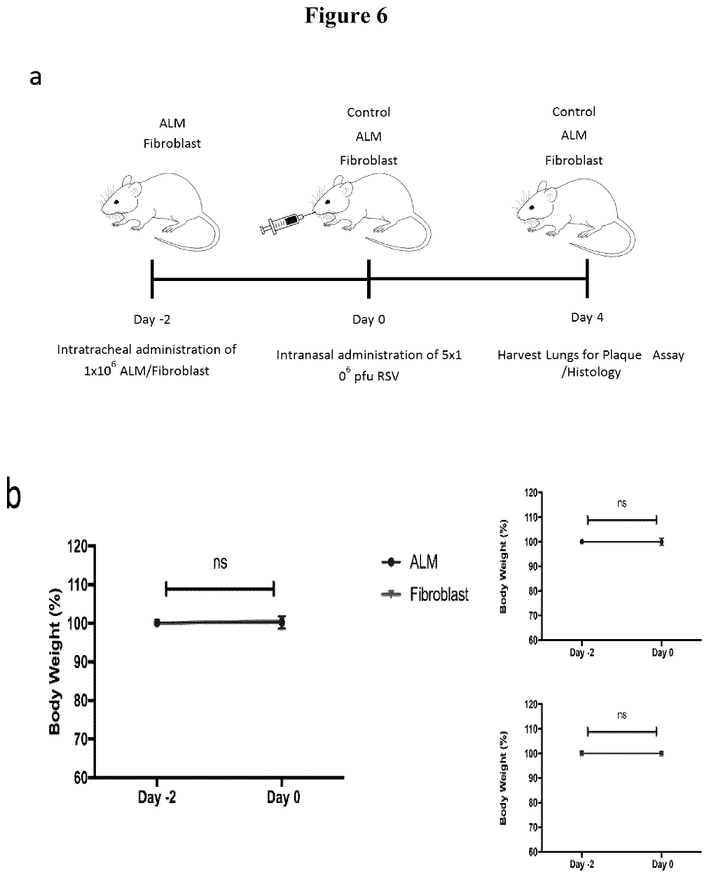

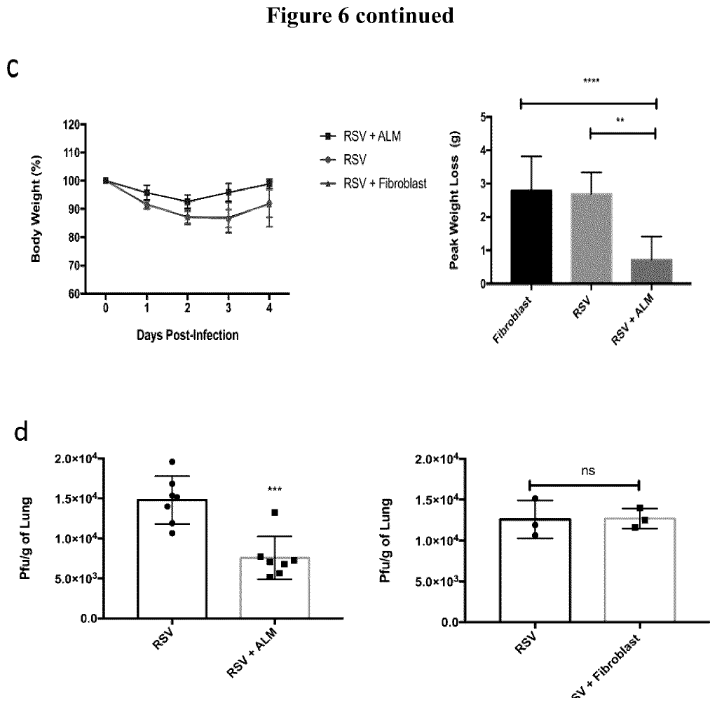

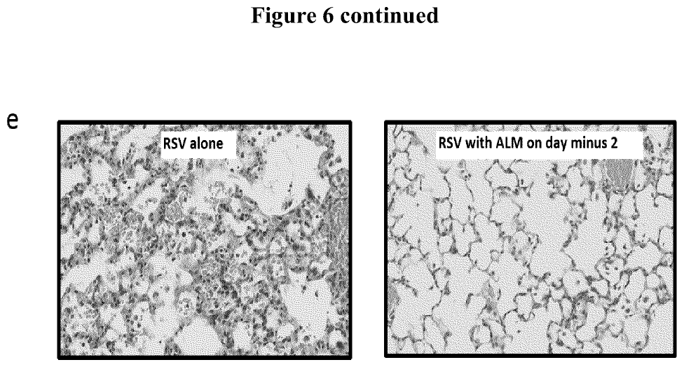

The potential for ALM cell therapy was investigated in an in vivo model of infection as summarized in FIG. 6(a). Briefly, Balb/c mice were intra-trachaelly instilled with 1×10⁶ ALMs or NIH3T3 cells two days prior to RSV infection. Body weights were measured for the two days following instillation and showed no significant differences (FIG. 6(b)). Mice were then given an intranasal inoculation of RSV-A2 two days after instillation of ALMs or NIH3T3 (at Day 0), and body weights were measured daily until Day 4.

Body weights measured following RSV inoculation showed a trend of weight loss peaking at day 3 in the untreated groups. ALM-instilled mice exhibit reduced body weight loss. For example, peak weight loss was about 2.69 g in RSV mice and only about 0.72 g in ALM treated mice as shown in FIG. 6 (c).

On Day 4, mice were euthanized and lungs were harvested for plaque assay or inflated and fixed for histological assessment. Lungs were homogenized and assayed for RSV viral tires by plaque forming assay over HEp-2 cells. ALM instillation significantly reduced viral titres. For example, viral titers in infected untreated tissue was found to be about 14786 pfu/g of lung in this experiment, while viral titers in ALM-treated tissue was about 7571 pfu/g of lung as shown in FIG. 6(d). NIH3T3 instillation did not affect viral titres.

For histological assessment, lungs were inflated to 20 cm H₂O pressure prior to formalin fixation. Processing and staining with hematoxylin and eosin were performed. Lung tissue from ALM-treated mice exhibited less inflammation and lung pathology. In particular, the lung tissue of ALM treated mice displayed less cellular infiltrate, less evidence of hemorrhage into the airway and less epithelial disruption than untreated tissue as shown in FIG. 6(e).

Relevant portions of references referred to herein are incorporated by reference.

1. A method of treating a respiratory viral infection in a mammal comprising administering to the mammal a therapeutically effective amount of alveolar-like macrophages, or an anti-viral factor produced by alveolar-like macrophages.

2. The method of claim 1, wherein the respiratory viral infection is caused by a virus of the Adenoviridae family, a virus of the Parvoviridae family, a virus of the Coronaviridae family, a virus of the Picornaviridae family, a virus of the Pneumoviridae family, a virus of the Orthomyxoviridae family or a virus of the Paramyxoviridae family.

3. The method of claim 2, wherein the virus is a respiratory syncytial virus.

4. The method of claim 1, wherein the alveolar-like macrophages express one or more markers selected from the group consisting of F4/80, EMR1, SiglecF, CD11c, CD68, CD169, CD163, AcLDL, CD45, CD11b, SIRPα, CD80, CD86 and CD206.

5. The method of claim 1, wherein the alveolar-like macrophages are prepared by culturing hemangioblasts in an alveolar macrophage-inducing medium comprising Granulocyte-Macrophage Colony-Stimulating Factor (GM-CSF) for a sufficient period of time.

6. The method of claim 5, wherein the alveolar macrophage-inducing medium additionally comprises one or more of Macrophage Colony-Stimulating Factor (M-CSF), IL-3, IL-6 and SCF.

7. The method of claim 6, wherein the alveolar macrophage-inducing medium comprises an amount of GM-CSF of about 10-100 ng/ml and an amount of M-CSF of about 10-100 ng/ml, and optionally, IL-3 in an amount in the range of about 10-100 ng/ml, IL-6 in an amount in the range of about 1-50 ng/ml and SCF in an amount in the range of about 10-100 ng/ml.

8. The method of claim 7, wherein the medium comprises GM-CSF and M-CSF in a 1:1 ratio.

9. The method of claim 7, wherein the medium comprises about 10-50 ng/ml of GM-CSF and about 10-50 ng/ml of M-CSF.

10. The method of claim 5, wherein the hemangioblasts are prepared from pluripotent stem cells.

11. The method of claim 1, wherein the alveolar-like macrophages are formulated for administration to the respiratory tract of the mammal.

12. The method of claim 11, wherein the alveolar-like macrophages are formulated for administration intra-tracheally or intranasally.

13. The method of claim 1, wherein the alveolar-like macrophages are formulated as a suspension in a medical-grade, physiologically acceptable carrier.

14. The method of claim 1, wherein the alveolar-like macrophages are formulated for administration by inhalation.

15. The method of claim 1, wherein the alveolar-like macrophages are administered to the respiratory tract of the mammal at a dosage in the range of about 10⁵ to 10¹⁰ cells.

16. An isolated anti-viral factor produced by alveolar-like macrophages.

17. The anti-viral factor of claim 16, which is produced on exposure of the alveolar-like macrophages to competent RSV.

18. The anti-viral factor of claim 16, which is resistant to UV radiation.

19. The anti-viral factor of claim 16, which reduces RSV infectivity against human epithelial cells.
